(19) United States
(12) Patent Application Publication
(10) Pub. No.: US 2021/0373030 A1
(43) Pub. Date: Dec. 2, 2021

(54) MARKER FOR DETERMING CRITICAL STAGE KIDNEY DISEASE

(71) Applicants: Kagami Inc., Ibaraki-shi, Osaka (JP); National University Corporation Kanazawa University, Kanazawa-shi, Ishikawa (JP)

(72) Inventors: Masashi MITA, Tokyo (JP); Takashi WADA, Ishikawa (JP); Kengo FURUICHI, Ishikawa (JP); Norihiko SAKAI, Ishikawa (JP); Yasunori IWATA, Ishikawa (JP); Shinji KITAJIMA, Ishikawa (JP); Yusuke NAKADE, Ishikawa (JP)

(73) Assignees: Kagami Inc., Ibaraki-shi, Osaka (JP); National University Corporation Kanazawa University, Kanazawa-shi, Ishikawa (JP)

(21) Appl. No.: 17/286,355

(22) PCT Filed: Oct. 17, 2019
(86) PCT No.: PCT/JP2019/040968

§ 371 Date: Aug. 4, 2021

(30) Foreign Application Priority Data
Oct. 17, 2018 (JP) 2018-196250

(51) Int. Cl. G01N 33/68; G01N 33/70

(52) U.S. Cl. CPC G01N 33/6812; G01N 33/70

(57) ABSTRACT

The present invention provides: a marker for determining critical stage kidney disease by using an indicator value based on the amount of D-alanine in the blood or the amount of D-alanine and L-alanine therein; a blood analysis method which uses said marker for patients undergoing surgery or intensive care; and a blood analysis system for determining critical stage kidney disease in patients undergoing surgery or intensive care.

### FIELD

The present invention relates to a marker for assessing critical phase kidney damage during surgery or intensive care, to an analysis method for determining critical phase kidney damage during surgery or intensive care, and to an analysis system for assessing critical phase kidney damage during surgery or intensive care.

### BACKGROUND

The purpose of surgery or intensive care medical treatment is to make use of highly advanced medical technology to restore or stabilize serious dysfunction of organ systems in the human body, thus providing life support. The role of the kidneys in the body is excretion of waste products, adjustment of blood pressure, and maintenance of homeostasis by controlling body fluids and ion regulation, and complications of kidney disease in surgery and intensive care can cause or amplify insufficiencies of other organs. A state of drastic reduction in renal function with serious multiple organ failure or sepsis associated with unstable circulatory dynamics in the critical phase of surgery or intensive care is referred to as acute kidney injury (AKI), and mortality rates have been reported to significantly increase with complications of AKI. With advances in medical care it has become common for surgery and intensive care to be provided for very elderly persons at high risk who previously could not be treated invasively, and this is one reason for the increase in critical phase AKI. It has been verified that AKI occurs in 40 to 60% of cases at intensive care unit (ICU) cohorts. While AKI is a pathology of the kidneys, however, its role in systemic diseases such as multiple organ failure and sepsis is also becoming noted, and it is usually treated by doctors other than kidney specialists.

It is recognized that AKI prognosis must be improved by earlier diagnosis and treatment intervention, and the RIFLE classification, as well as AKIN criteria or KDIGO criteria, have been proposed. Serum creatinine used in these classifications and criteria is known to have lower sensitivity to increase in early AKI. Furthermore, since serum creatinine is significantly affected by muscle mass and is particularly unstable in patients with emaciation or prolonged bed rest, as is common with the very elderly, it cannot be considered to be a specific diagnostic marker (NPL 1), and it is therefore desirable to obtain early diagnosis using multiple biomarkers with different sensitivities and specificities, such as NGAL and L-FABP which are recently being implemented.

Conventionally, D-amino acids had been considered to be absent from the mammalian bodies but have since been shown to be present in various tissues and to carry out physiological functions. It has been shown that, among D-amino acids in human blood, the amounts of D-serine, D-alanine, D-proline, D-glutamic acid and D-aspartic acid in blood can serve as diagnostic markers for kidney disease, since they correlate with serum creatinine levels (NPL 3, NPL 4, NPL 5). It has also been disclosed that one or more amino acids selected from the group consisting of D-serine, D-threonine, D-alanine, D-asparagine, D-allothreonine, D-glutamine, D-proline and D-phenylalanine can serve as pathology marker values for kidney disease (PTL 1). It has been shown that D-serine in mouse blood increases with ischemia reperfusion treatment and that D-serine in mouse urine decreases with ischemia reperfusion treatment (PTL 2, NPL 6). These publications deal with ischemia reperfusion treatment in a model of acute kidney injury in mice, but the mice suffering from nephropathy in the treatment die without improvement in renal function, and such mice therefore do not serve as models accurately reflecting human AKI pathology, where renal function is reversible. In addition, prognosis and prediction of chronic kidney disease is carried out based on analysis that distinguishes the optical isomers of amino acids in blood (NPL 7), but no biomarkers have been found with analysis of optical isomers of amino acids in blood for human AKI.

### CITATION LIST

### Patent Literature

[PTL 1] International Patent Publication No. 2013/140785
[PTL 2] International Patent Publication No. 2015/087985

### Non Patent Literature

[NPL 1] Slocum, J. L. et al., Transl Res. 159:277 (2012)
[NPL 2] KDIGO 2012 Clinical Practice Guideline for the Evaluation and Management of Chronic Kidney Disease, Kidney International Supplements 1 (2013)
[NPL 3] Fukushima, T. et al., Biol. Pharm. Bull. 18: 1130(1995)
[NPL 4] Nagata Y. Viva Origino Vol. 18(No. 2) (1990), 15th Lecture Meeting Abstracts
[NPL 5] Ishida et al., Kitasato Medical Journal 23:51-62(1993)
[NPL 6] Sasabe J. et al., PLOS ONE (2014) vol. 9, Issue 1, e86504
[NPL 7] Kimura T. et al., Scientific Reports 6:26137 DOI:10.1038/srep26137

### SUMMARY

### Technical Problem

It is an object of the invention to provide a diagnostic marker for critical phase kidney damage that can replace or supplement the existing diagnostic markers for acute kidney injury, such as serum creatinine.

### Solution to Problem

The present inventors have searched for biomarkers that can be used for diagnosis of critical phase kidney damage in patients under medical treatment at intensive care units and have found, surprisingly, that markers based on D-alanine levels in blood or D-alanine and L-alanine levels in blood exhibit very high correlation with serum creatinine. This has led to the discovery that markers based on blood D-alanine levels and blood D-alanine and L-alanine levels can serve as diagnostic markers for critical phase kidney damage, on the basis of which the present invention has been completed. The present invention thus relates to the following:

[1] A marker for assessment of critical phase kidney damage, which is a marker value based on blood D-alanine level or D-alanine level and L-alanine level.

[2] The marker according to [1] above, wherein the marker value based on D-alanine level and L-alanine level is a ratio or percentage.

[3] The marker according to [1] or [2] above, wherein critical phase kidney damage is assessed for a patient under surgery or intensive care.

[4] The marker according to [1] above, wherein the patient under surgery or intensive care is in a state selected from the group consisting of dehydration, nephrotic syndrome, glomerular nephritis, rapidly progressive glomerulonephritis and blood pressure drop.

[5] A blood analysis method for a patient under surgery or intensive care, wherein the blood analysis method comprises:

a step of measuring blood D-alanine level or D-alanine level and L-alanine level, and

a step of associating a marker value based on the D-alanine level or the D-alanine level and L-alanine level with critical phase kidney damage.

[6] The blood analysis method according to [5] above, wherein the marker value based on D-alanine level and L-alanine level is a ratio or percentage.

[7] The blood analysis method according to [5] or [6] above, wherein the patient under surgery or intensive care is in a state selected from the group consisting of dehydration, nephrotic syndrome, glomerular nephritis, rapidly progressive glomerulonephritis and blood pressure drop.

[8] The blood analysis method according to any one of [5] to [7] above, which is for specifying the stage or pathological condition in the critical phase by combining renal function markers.

[9] The blood analysis method according to [8] above, wherein the renal function marker is at least one marker selected from the group consisting of urine NGAL, blood NGAL, urine IL-18, urine KIM-1, urine L-FABP, blood creatinine, urine creatinine, blood cystatin C, urine protein, urine albumin, urine β2-MG, urine α1-MG, urine NAG, eGFR (creatinine, cystatin C) and blood urea nitrogen.

[10] A method for diagnosing critical phase kidney damage in a patient under surgery or intensive care, and treating, wherein the method comprises:

a step of measuring blood D-alanine level or D-alanine level and L-alanine level,

a step of diagnosing critical phase kidney damage from a marker value based on the D-alanine level or the D-alanine level and L-alanine level, and

a step of carrying out treatment intervention for the patient suffering from critical phase kidney damage.

[11] The method according to [10] above, wherein the treatment intervention is one or more selected from the group consisting of lifestyle habit improvement, dietary guidance, effective circulating blood volume or blood pressure maintenance, renal function alternative therapy, blood pressure management, blood sugar level management, immune management and lipid management.

[12] The method according to [10] or [11] above, wherein the treatment intervention includes administration to a subject of one or more drugs selected from the group consisting of diuretic drugs, medullary fluids, isotonic crystalline liquids, infusions, hypertensive agents, calcium antagonists, angiotensin converting enzyme inhibitors, angiotensin receptor antagonists, sympatholytic drugs, SGLT2 inhibitors, sulfonylurea drugs, thiazolidine drugs, biguanide drugs, α-glucosidase inhibitors, glinide drugs, insulin formulations, NRF2 activators, immunosuppressive agents, statins, fibrates, anemia treatments, erythropoietin formulations, HIF-1 inhibitors, iron agents, electrolyte regulators, calcium receptor agonists, phosphorus adsorbents, uremic toxin adsorbents, DPP4 inhibitors, EPA formulations, nicotinic acid derivatives, cholesterol transporter inhibitors and PCSK9 inhibitors.

[13] A blood analysis system for determining critical phase kidney damage for a patient under surgery or intensive care, comprising a storage unit, an analytical measurement unit, a data processing unit and a pathological information output unit, wherein:

the storage unit stores a threshold for assessment of critical phase kidney damage,

the analytical measurement unit separates and quantifies the blood D-alanine level or the D-alanine level and L-alanine level,

the data processing unit compares the marker value for D-alanine level or for D-alanine level and L-alanine level of the inpatient with the threshold stored in the storage unit, and assesses critical phase kidney damage, and

the pathological information output unit outputs information relating to critical phase kidney damage.

[14] The blood analysis system according to [13] above, wherein the marker value based on D-alanine level and L-alanine level is a ratio or percentage.

[15] The blood analysis system according to [13] or [14] above, wherein the patient under surgery or intensive care is in a state selected from the group consisting of dehydration, nephrotic syndrome, glomerular nephritis, rapidly progressive glomerulonephritis and blood pressure drop.

[16] A program which causes an information processing device comprising an input unit, an output unit, a data processing unit and a storage unit to determine critical phase kidney damage, wherein the program includes commands to cause the information processing device to:

cause the storage unit to store a calculation formula for a marker value inputted through the input unit, and a threshold for the marker value,

cause the storage unit to store a blood level of D-alanine or of D-alanine and L-alanine that has been inputted through the input unit,

cause the data processing unit to read the stored D- and L-alanine blood levels and the calculation formula for the marker value and calculate the marker value, and cause the storage unit to store them, and

cause the data processing unit to read the stored marker value and the marker value threshold and compare the marker value with the threshold, and to output the presence or absence of critical phase kidney damage to the output unit.

### Advantageous Effects of Invention

The present invention allows assessment of critical phase kidney damage.

### BRIEF DESCRIPTION OF DRAWINGS

FIG. 1A is a scatter plot showing the correlation between blood D-alanine/L-alanine ratio and serum creatinine, and FIG. 1B is a scatter plot showing the correlation between blood D-alanine/L-alanine ratio and estimated glomerular filtration rate (eGFR) determined from serum creatinine.

FIG. 2A is a scatter plot showing the correlation between blood D-alanine level and serum creatinine, and FIG. 2B is a scatter plot showing the correlation between blood D-alanine level and estimated glomerular filtration rate (eGFR) determined from serum creatinine.

FIG. 3 is a block diagram of a sample analysis system of the invention.

FIG. 4 is a flow chart showing an example of operation for determining glomerular filtration rate by the program of the invention.

### DESCRIPTION OF EMBODIMENTS

The present invention relates to a marker for assessing critical phase kidney damage which is a marker value based on blood D-alanine level or on D-alanine level and L-alanine level, to a blood analysis method for inpatients or for surgery or intensive care, to a blood analysis system that outputs information relating to critical phase kidney damage, and to an operating program.

Critical phase kidney damage is a condition whose vital prognosis can be improved by controlling symptoms such as uremia via renal function alternative therapy or blood pressure management, or by drug intervention, against acute loss of renal function. Critical phase kidney damage may also refer to kidney damage during surgery or intensive care, or kidney damage as a complication of multiple organ failure or sepsis.

Patients admitted into intensive care unit (ICU), for heart disease (such as cardiac failure, arrhythmia, valve disease, coronary disease or aortic disease), gastrointestinal disease (such as esophageal cancer, pancreatic cancer or liver cancer), encephalopathy (such as cerebral infarction, intracerebral hemorrhage, subarachinoid hemorrhage, convulsion, epilepsy, brain tumor or cerebral aneurysm), cervical spine disease, kidney transplant, pneumonia or sepsis, also experience sudden dehydration or blood pressure drop, and the kidney damage may lead to or amplify other organ insufficiencies. Therefore, acute kidney injury (AKI) in intensive care, while occurring as damage to a single organ, can lead to the complication of multiple organ failure, or it may occur as a symptom of multiple organ failure.

Most critical phase kidney damage is diagnosed by decreased urinary volume and elevated serum creatinine, according to KDIGO guidelines. The actual acute kidney disease (AKI) classification is listed in the following table.

TABLE 1
AKI severity classification

| Stage | Serum creatinine | Urine volume |
|---|---|---|
| 1 | 1.5-1.9 times base value, or | Reduction to below 0.5 mL/kg/hr |
| | increase of ≥0.3 mg/dL | for ≥6-12 hours |
| 2 | 2.0-2.9 times base value | Reduction to below 0.5 mL/kg/hr |
| | | for ≥12 hours |
| 3 | 3 times base value or increase | Reduction to below 0.3 mL/kg/hr |
| | of ≥4 mg/dL, or initiation | for ≥24 hours, or no urine |
| | of renal replacement therapy, | for ≥12 hours. |
| | or reduction to eGFR <35 | |
| | mL/min/1.73 m² | |
| | (for less than 18 yrs of age) | |

Serum creatinine is a metabolite of creatine phosphate in the muscle, and its amount is known to depend on muscle mass. With onset of acute kidney injury in which production and excretion are not steady, serum creatinine does not reflect changes in renal function accurately or with sensitivity, and it has been noted that it does not increase in early pathology. It has been conjectured that one reason for the failure of various treatment intervention tests to date is the lack of precision in AKI diagnosis based on serum creatinine standards.

The marker values based on blood D-alanine level or D-alanine level and L-alanine level according to the invention have shown high correlation with serum creatinine in patients under intensive care. This indicates that the marker values can serve as markers for assessment of critical phase kidney damage. In normally healthy people, blood D-alanine levels are strictly controlled by metabolic systems (synthesis and decomposition) involving enzymes such as alanine racemase and D-amino acid oxidase, but are known to fluctuate with changes in renal glomerular filtration or reabsorption power, and they can therefore serve as sensitive markers based on a different mechanism from serum creatinine. Since the vital prognosis of critical phase kidney damage is greatly affected by early appropriate intervention, and it is rarely dealt with by kidney specialists, it would be useful for diagnosis to be panelized using multiple markers with high sensitivity and different variation mechanisms.

The causes of acute kidney injury are largely grouped into prerenal, renal and postrenal causes. Prerenal causes, being systemic disease, refer to those resulting from reduced blood flow to the kidneys, and can occur due to dehydration, shock, burn, massive hemorrhage, blood pressure drop, congestive heart failure, hepatic cirrhosis or renal artery stenosis. Renal causes arise from the kidneys themselves, and include blood flow disturbance in the kidneys, glomerular disorder and renal tubular/interstitial disorder. Diseases caused by impaired blood flow to the kidneys include bilateral renal infarction, renal artery thrombus, disseminated intravascular coagulation syndrome, thrombotic thrombocytopenic purpura and hemolytic uremia syndrome. Glomerular diseases include nephrotic syndrome, acute glomerular nephritis, rapidly progressive glomerulonephritis, lupus nephritis (systemic lupus erythematosus), ANCA-associated vasculitis and polyarteritis nodosa. All of these causes can be factors that lead to critical phase kidney damage, but the major causes of critical phase kidney damage in particular are prerenal causes such as dehydration, blood pressure drop, hemorrhage and cardiac failure-induced ischemia, and renal causes such as nephrotic syndrome, acute glomerular nephritis, rapidly progressive glomerulonephritis and lupus nephritis.

A marker value used for the invention may be the blood D-alanine level itself, or a marker value based on D-alanine level and L-alanine level. A marker value based on D-alanine level and L-alanine level may be, for example, the ratio of a D-alanine level and L-alanine level (D-Ala/L-Ala or L-Ala/D-Ala) or the percentage of a D-alanine level (such as D-Ala/(D-Ala+L-Ala)×100), although addition, subtraction, integration and/or division of any constant or any variable such as age, body weight, gender, BMI and eGFR may also be included in the formula so long as critical phase kidney damage can be assessed. Using a quantity ratio with an optical isomer of an amino acid as the marker value is advantageous as it eliminates the need for correction based on the amount or volume of sample.

By comparing the marker value of the invention with a preset threshold it is possible to assess critical phase kidney damage. Several levels of threshold can also be used to determine the stage of pathology. Thresholds can be appropriately set by large scale examinations. They may also be set to correspond to currently used standards for serum creatinine or estimated glomerular filtration rate. From the viewpoint of more sensitive assessment of critical phase kidney damage, it is preferred to carry out large scale examination of marker values for D-alanine levels, or for D-alanine levels and L-alanine levels.

According to the invention the target of assessment of critical phase kidney damage may be any subject, but from the viewpoint of assessing critical phase kidney damage it is preferably a patient under surgery or intensive care. Patients under intensive care may be patients exhibiting serious symptoms in a ward, or emergency patients in need of continuous management, or patients in need of advanced management after surgery. Blood samples can be obtained at any time including before surgery, during surgery or after surgery. Blood samples may also be obtained periodically.

One mode of the invention relates to a blood analysis method for surgery or intensive care, wherein the blood analysis method comprises:

a step of measuring blood D-alanine level or D-alanine level and L-alanine level, and

a step of associating a marker value based on the D-alanine level or the D-alanine level and L-alanine level with critical phase kidney damage.

The analysis method of the invention may be used to provide preliminary data for diagnosis by a physician, and may therefore be considered a preliminary method to diagnosis. Using such preliminary data allows a physician to diagnosis acute kidney injury, and the analysis method may also be carried out by a medical assistant who is not a physician, or by an analytical institution. The analysis method of the invention may therefore be considered to be a method that is preliminary to diagnosis. The analysis method may also include a step of associating marker values with the pathology of critical phase kidney damage. Such an analysis method may be carried out by an analysis company or analysis technician, to provide results associated with the pathology of kidney damage. More preferably, the analysis is periodical for an inpatient, and especially a patient under surgery or intensive care.

Another mode of the invention can specify the stage or pathological condition of a critical phase, by combining a marker of the invention with a renal function marker. The renal function marker to be used in combination may be a marker that is known or under development, examples of which include one or more markers selected from the group consisting of urine NGAL, blood NGAL, urine IL-18, urine KIM-1, urine L-FABP, blood creatinine, urine creatinine, blood cystatin C, urine protein, urine albumin, urine β2-MG, urine α1-MG, urine NAG, eGFR (creatinine, cystatin C) and blood urea nitrogen. By using multiple markers, it is possible to properly assess the initial stage of kidney damage, the expansion period of kidney damage, the persistent stage of kidney damage and the repair stage of kidney damage.

The step of associating a marker value with critical phase kidney damage may be a step of comparing the threshold for a marker value based on D-alanine level, or D-alanine level and L-alanine level, with a calculated marker value, and if the threshold is exceeded, determining that critical phase kidney damage is present.

A blood amino acid level according to the invention is the amino acid level determined by separating the individual optical isomers, and it may refer to the amino acid level in a specific blood volume, and expressed as a concentration. The blood amino acid level is measured as the amount in a sample of blood that has been treated by centrifugal separation, sedimentation separation or other pretreatment for analysis. Therefore, the amino acid level can be measured as the amount in a blood sample derived from sampled whole blood, serum or blood plasma. For analysis using HPLC, for example, a specific optical isomer of an amino acid in a predetermined amount of blood may be represented in a chromatogram, and the peak heights, areas and shapes may be quantified by analysis based on standard sample comparison and calibration. With an enzyme method, the amino acid concentration can be calculated by quantitative analysis using a standard calibration curve.

The D-alanine and L-alanine levels may be measured by any method, such as chiral column chromatography, or measurement using an enzyme method, or quantitation by an immunological method using a monoclonal antibody that distinguishes between optical isomers of amino acids. Measurement of the D-alanine and L-alanine levels in a sample according to the invention may be carried out using any method well known to those skilled in the art. Examples include chromatographic and enzyme methods (Y. Nagata et al., Clinical Science, 73 (1987), 105. Analytical Biochemistry, 150 (1985), 238., A. D'Aniello et al., Comparative Biochemistry and Physiology Part B, 66 (1980), 319. Journal of Neurochemistry, 29 (1977), 1053., A. Berneman et al., Journal of Microbial & Biochemical Technology, 2 (2010), 139., W. G. Gutheil et al., Analytical Biochemistry, 287 (2000), 196., G. Molla et al., Methods in Molecular Biology, 794 (2012), 273., T. Ito et al., Analytical Biochemistry, 371 (2007), 167.), antibody methods (T. Ohgusu et al., Analytical Biochemistry, 357 (2006), 15), gas chromatography (GC) (H. Hasegawa et al., Journal of Mass Spectrometry, 46 (2011), 502., M. C. Waldhier et al., Analytical and Bioanalytical Chemistry, 394 (2009), 695., A. Hashimoto, T. Nishikawa et al., FEBS Letters, 296 (1992), 33., H. Bruckner and A. Schieber, Biomedical Chromatography, 15 (2001), 166., M. Junge et al., Chirality, 19 (2007), 228., M. C. Waldhier et al., Journal of Chromatography A, 1218 (2011), 4537), capillary electrophoresis methods (CE) (H. Miao et al., Analytical Chemistry, 77 (2005), 7190., D. L. Kirschner et al., Analytical Chemistry, 79 (2007), 736., F. Kitagawa, K. Otsuka, Journal of Chromatography B, 879 (2011), 3078., G. Thorsen and J. Bergquist, Journal of Chromatography B, 745 (2000), 389.), and high-performance liquid chromatography (HPLC) (N. Nimura and T. Kinoshita, Journal of Chromatography, 352 (1986), 169., A. Hashimoto et al., Journal of Chromatography, 582 (1992), 41., H. Bruckner et al., Journal of Chromatography A, 666 (1994), 259., N. Nimura et al., Analytical Biochemistry, 315 (2003), 262., C. Muller et al., Journal of Chromatography A, 1324 (2014), 109., S. Einarsson et al., Analytical Chemistry, 59 (1987), 1191., E. Okuma and H. Abe, Journal of Chromatography B, 660 (1994), 243., Y. Gogami et al., Journal of Chromatography B, 879 (2011), 3259., Y. Nagata et al., Journal of Chromatography, 575 (1992), 147., S. A. Fuchs et al., Clinical Chemistry, 54 (2008), 1443., D. Gordes et al., Amino Acids, 40 (2011), 553., D. Jin et al., Analytical Biochemistry, 269 (1999), 124., J. Z. Min et al., Journal of Chromatography B, 879 (2011), 3220., T. Sakamoto et al., Analytical and Bioanalytical Chemistry, 408 (2016), 517., W. F. Visser et al., Journal of Chromatography A, 1218 (2011), 7130., Y. Xing et al., Analytical and Bioanalytical Chemistry, 408 (2016), 141., K. Imai et al., Biomedical Chromatography, 9 (1995), 106., T. Fukushima et al., Biomedical Chromatography, 9 (1995), 10., R. J. Reischl et al., Journal of Chromatography A, 1218 (2011), 8379., R. J. Reischl and W. Lindner, Journal of Chromatography A, 1269 (2012), 262., S. Karakawa et al., Journal of Pharmaceutical and Biomedical Analysis, 115 (2015), 123.).

The separative analysis system for optical isomers according to the invention may be a combination of multiple separative analysis methods. More specifically, the D-/L-amino acid level in a sample can be measured using an optical isomer analysis method comprising a step of passing a sample containing a component with optical isomers through a first column filler as the stationary phase, together with a first liquid as the mobile phase, to separate the components in the sample, a step of separately holding each of the components in the sample in a multi loop unit, a step of passing each of the components in the sample that are separately held in the multi loop unit through a flow channel in a second column filler having an optically active center, as the stationary phase, together with a second liquid as the mobile phase, to separate the optical isomers among each of the sample components, and a step of detecting the optical isomers in each of the sample components (Japanese Patent No. 4291628). In HPLC analysis, D- and L-amino acids are sometimes pre-derivatized with a fluorescent reagent such as o-phthalaldehyde (OPA) or 4-fluoro-7-nitro-2,1,3-benzooxadiazole (NBD-F), or diastereomerized using an agent such as N-tert-butyloxycarbonyl-L-cysteine (Boc-L-Cys) (Hamase, K. and Zaitsu, K., Bunseki Kagaku, Vol. 53, 677-690(2004)). Alternatively, the D-amino acids may be measured by an immunological method using a monoclonal antibody that distinguishes optical isomers of amino acids, such as a monoclonal antibody that specifically binds to D-alanine or L-alanine. When the total of the D-form and L-form is to be used as the marker it is not necessary to separate the D-form and L-form, allowing the amino acids to be analyzed without separating the D-form and L-form. In such cases as well, separation and quantitation may be carried out using an enzyme method, antibody method, GC, CE or HPLC.

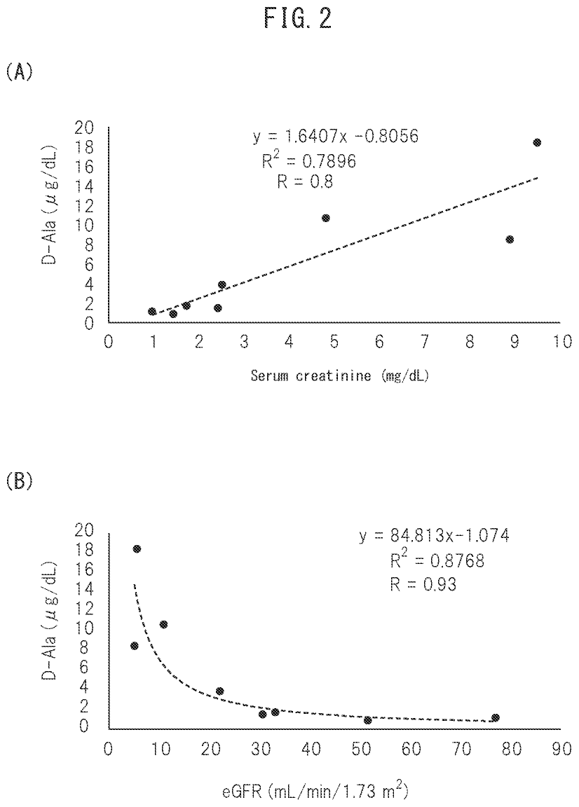

FIG. 2 is a block diagram of a sample analysis system of the invention. The sample analysis system 10 of the invention shown in FIG. 2 is constructed so as to allow the analysis method and examination method of the invention to be carried out. The sample analysis system 10 comprises a storage unit 11, an input unit 12, an analytical measurement unit 13, a data processing unit 14 and an output unit 15, and allows analysis of blood samples and output of information relating to critical phase kidney damage. More specifically, the sample analysis system 10 of the invention is a blood analysis system wherein:

the storage unit 11 stores a marker value threshold for assessment of critical phase kidney damage that has been inputted through the input unit 12,

the analytical measurement unit 13 separates and quantifies a blood D-alanine level or a D-alanine level and L-alanine level,

the data processing unit 14 calculates a marker value based on the blood D-alanine level or the D-alanine level and L-alanine level,

the data processing unit 14 discriminates information for critical phase kidney damage based on comparison with the threshold stored in the storage unit 11, and

the output unit 15 outputs information relating to critical phase kidney damage.

The storage unit 11 has a portable storage device which may be a memory device such as a RAM, ROM or flash memory, a fixed disk device such as a hard disk drive, or a flexible disk or optical disk. The storage unit stores data measured by the analytical measurement unit, data and instructions inputted by the input unit, and results of computation processing by the data processing unit, as well as the computer program and database to be used for processing by the information processing equipment. The computer program may be a computer readable recording medium such as a CD-ROM or DVD-ROM, or it may be installed via the internet. The computer program is installed in the storage unit using a commonly known setup program, for example.

The input unit 12 is an interface and also includes operating devices such as a keyboard and mouse. This allows the input unit to input data measured by the analytical measurement unit 13 and instructions for computation processing to be carried out by the data processing unit 14. When the analytical measurement unit 13 is external, for example, the input unit 12 may also include an interface unit allowing input of measured data through a network or storage medium, separately from the operating device.

The analytical measurement unit 13 carries out a step of measuring the amount of D-form and L-form of an amino acid in a blood sample. The analytical measurement unit 13 therefore has a construction allowing separation and measurement of the D-forms and L-forms of amino acids. The amino acids may be analyzed one at a time, or some or all of the amino acid types may be analyzed at once. With no intention to be limitative, the analytical measurement unit 13 may be a high-performance liquid chromatography system comprising a sample introduction inlet, an optical resolution column and a detector, for example. The analytical measurement unit 13 may be constructed separately from the sample analysis system, and measured data may be inputted through the input unit 12 using a network or storage medium. The analytical measurement unit 13 of the invention may further comprise a sample acquisition unit, with a sample being acquired periodically by the sample acquisition unit and the acquired sample being provided to the analytical measurement unit.

The data processing unit 14 carries out various computation processing operations on the data measured by the analytical measurement unit 13 and stored in the storage unit 11, based on a program stored in the storage unit. Computation processing is carried out by a processor or CPU inside the data processing unit. The processor or CPU includes a functional module that controls the analytical measurement unit 13, input unit 12, storage unit 11 and output unit 15, the functional module performing various control operations. Each of the units may be constructed by independent integrated circuits, microprocessors and firmware. The data processing unit 14 calculates the marker value based on D-alanine level or on D-alanine level and L-alanine level according to a formula, compares it with a threshold for the marker value stored in the storage unit, and assesses the state of critical phase kidney damage.

The output unit 15 is configured to output the presence or absence of critical phase kidney damage as the result of computation processing by the data processing unit. The output unit 15 may be output means such as a display device with a liquid crystal display that directly displays the computation processing results, or a printer, or it may be an interface unit for output to an external memory unit or output to a network.

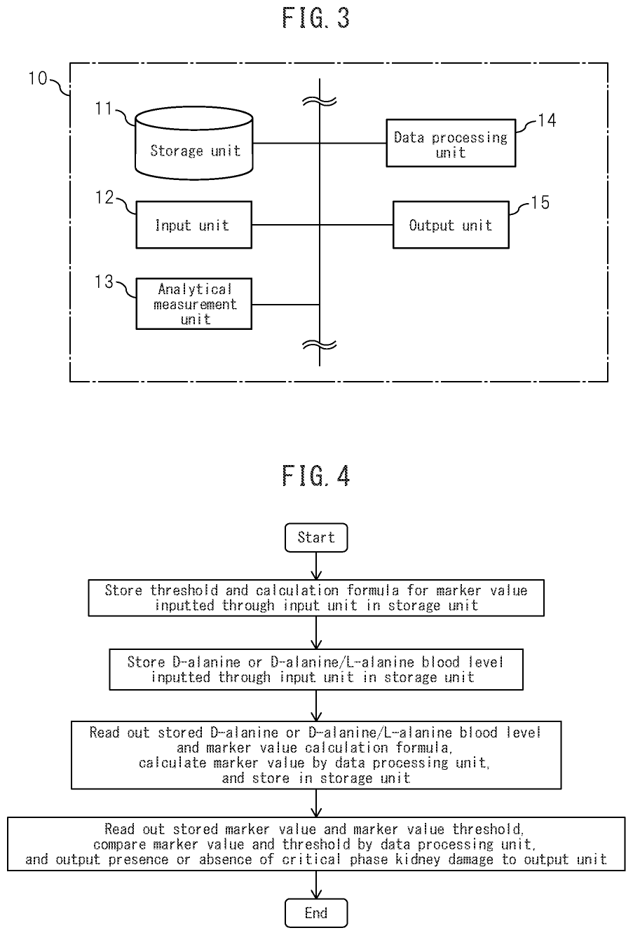

Yet another mode of the invention relates to a program that operates the blood analysis system and information processing device. FIG. 3 is a flow chart showing an example of operation for output of the presence or absence and severity of critical phase kidney damage to a program. Specifically, the program of the invention is a program that determines glomerular filtration rate in an information processing device comprising an input unit, output unit, data processing unit and storage unit. The program of the invention includes commands to cause the information processing device to:

cause the storage unit to store a calculation formula for a marker value inputted through the input unit, and a threshold for the marker value,

cause the storage unit to store a blood level of D-alanine or of D-alanine and L-alanine that has been inputted through the input unit,

cause the data processing unit to read the stored blood levels of D- and L-alanine and the calculation formula for the marker value and calculate the marker value, and cause the storage unit to store them, and

cause the data processing unit to read the stored marker value and the marker value threshold and compare the marker value with the threshold, and to output the presence or absence and severity of critical phase kidney damage to the output unit. The program of the invention may be stored in a storage medium, or it may be provided via electronic transmission such as the internet or a LAN.

When the information processing device comprises an analytical measurement unit, it may include a command for causing the information processing device to take the value for the blood sample measured by the analytical measurement unit and store it in the storage unit, instead of having the D-alanine level value inputted from an input unit.

According to the invention, when the subject has been clearly shown to be suffering from critical phase kidney damage, a biomarker may be monitored to determine treatment policy and assess its therapeutic effect. When onset of critical phase kidney damage has been assessed, treatment intervention may be initiated for, but not necessarily only for, maintaining effective circulating blood volume and blood pressure. When a nephrotoxic drug has been administered, administration of the drug may be suspended. Diuretics, medullary fluids, isotonic crystalline liquids, infusions or hypertensive agents (such as arterenol, synephrine, phenylephrine, methoxamine and mephentermine) may also be administered in order to maintain effective circulating blood volume and blood pressure. Other treatment interventions include guidance for lifestyle habit improvement, dietary guidance, blood pressure management, anemia management, electrolyte management, uremia management, blood sugar level management, immune management or lipid management, or drug therapy. Lifestyle habit improvement may be a recommendation to stop smoking or to reduce the BMI value to below 25. Dietary guidance may be salt or protein restriction. Blood pressure management may involve general management or administration of an antihypertensive drug, to reach below 130/80 mmHg. Antihypertensive drugs include diuretic drugs (thiazide diuretics such as trichlormethiazide, benzylhydrochlorothiazide and hydrochlorothiazide, thiazide-like diuretics such as meticrane, indapamide, tribamide and mefluside, loop diuretics such as furosemide, and potassium-sparing diuretics and aldosterone antagonists such as triamterene, spironolactone and eplerenone), calcium antagonists (dihydropyridine-based antagonists such as nifedipine, amlodipine, efonidipine, cilnidipine, nicardipine, nisoldipine, nitrendipine, nilvadipine, barnidipine, felodipine, benidipine, manidipine, azelnidipine and aranidipine, benzodiazepine-based antagonists, and diltiazem), angiotensin converting enzyme inhibitors (such as captopril, enalapril, acelapril, delapril, cilazapril, lisinopril, benazepril, imidapril, temocapril, quinapril, trandolapril and perindopril erbumine), angiotensin receptor antagonists (angiotensin II receptor antagonists such as losartan, candesartan, valsartan, telmisartan, olmesartan, irbesartan and azilsartan), and sympatholytic drugs (n-blockers, such as atenolol, bisoprolol, betaxolol, metoprolol, acebutolol, celiprolol, propranolol, nadolol, carteolol, pindolol, nipradilol, amosulalol, arotinolol, carvedilol, labetalol, bevantolol, urapidil, terazosin, prazosin, doxazosin and bunazosin). Erythropoietin formulations, iron agents and HIF-1 inhibitors are used as anemia treatments. Calcium receptor agonists (such as cinacalcet and etelcalcetide) and phosphorus adsorbents are used as electrolyte regulators. Active carbon is used as a uremic toxin adsorbent. Blood glucose level is managed to Hba1c of <6.9%, and in some cases a hypoglycemic agent is administered. Hypoglycemic agents that are used include SGLT2 inhibitors (such as ipragliflozin, dapagliflozin, luseogliflozin, tofogliflozin, canagliflozin and empagliflozin), DPP4 inhibitors (such as sitagliptin phosphate, vildagliptin, saxagliptin, alogliptin, linagliptin, teneligliptin, trelagliptin, anagliptin, omarigliptin), sulfonylurea agents (such as tolbutamide, acetohexamide, chlorpropamide, glyclopyramide, glibenclamide, gliclazide and glimepiride), thiazolidine agents (such as pioglitazone), biguanide agents (such as metformin and buformin), α-glucosidase inhibitors (such as acarbose, voglibose and miglitol), glinide agents (such as nateglinide, mitiglinide and repaglinide), insulin formulations and NRF2 activators (such as bardoxolonemethyl). Immunosuppressive agents (such as steroids, tacrolimus, anti-CD20 antibody, cyclohexamide and mycophenolate mofetil (MMF)) are used for immune management. Lipid management includes management to lower LDL-C to below 120 mg/dL, or in some cases dyslipidemia treatments are used, including statins (such as rosuvastatin, pitavastatin, atorvastatin, cerivastatin, fluvastatin, simvastatin, pravastatin, lovastatin and mevastatin), fibrates (such as clofibrate, bezafibrate, fenofibrate and clinofibrate), nicotinic acid derivatives (such as tocopherol nicotinate, nicomol and niceritrol), cholesterol transporter inhibitors (such as ezetimibe), PCSK9 inhibitors (such as evolocumab) and EPA formulations. All of these drugs may be used as single dosage forms for mixtures.

When renal function impairment is notable enough to pose a risk for vital prognosis, renal replacement therapy such as peritoneal dialysis, hemodialysis, continuous hemodialysis filtration, blood apheresis (such as blood plasma exchange or blood plasma adsorption) or kidney transplant may also be carried out.

All of the publications mentioned throughout the present specification are incorporated herein in their entirety by reference.

The examples of the invention described below are intended to serve merely as illustration and do not limit the technical scope of the invention. The technical scope of the invention is limited solely by the description in the Claims. Modifications of the invention, such as additions, deletions or substitutions to the constituent features of the invention, are possible so long as the gist of the invention is maintained.

### EXAMPLES

### Materials and Methods

### Materials

Amino acid standard samples and HPLC-grade acetonitrile were purchased from Nacalai Tesque, Inc. (Kyoto). HPLC-grade methanol, trifluoroacetic acid and boric acid were purchased from Wako Pure Chemical Industries, Ltd. (Osaka). The water was purified using a Milli-Q gradient A10 system.

### Pool of Patients

Blood samples were acquired from patients admitted to Kanazawa University Hospital from 2013 to 2017, who suffered from acute kidney disease (AKI) and had received intensive care. Patients undergoing therapy with immunosuppressive agents or antibiotics were excluded. Table 2 shows the clinical information for the acute kidney disease patients. All of the patients were examined for serum creatinine, urine protein, urine occult blood and diabetes at baseline and during AKI. The blood D-Ala concentration and D-Ala/L-Ala ratio were measured, using the following method for measuring D-amino acids. All of the patients had good vital prognosis with treatment intervention during AKI.

TABLE 2
Clinical features of critical phase patients

| Patient No. | | 1 | 2 | 3 | 4 | 5 | 6 | 7 | 8 |
|---|---|---|---|---|---|---|---|---|---|
| Gender | | M | M | M | M | M | M | M | M |
| Age | | 28 | 58 | 78 | 25 | 57 | 21 | 42 | 56 |
| AKI stage by KDIGO category | | 1 | 1 | 1 | 2 | 2 | 3 | 3 | 3 |
| Renal function | Cr | 0.70 | 1.12 | 4.95 | 0.69 | 1.05 | 0.65 | 1.07 | 1.18 |
| at baseline | eGFR | 110.1 | 53.4 | 9.7 | 115.6 | 57.6 | 129.7 | 61.6 | 51.0 |
| Renal function | Cr | 1.35 | 2.11 | 9.42 | 2.30 | 3.84 | 2.43 | 10.87 | 5.56 |
| during AKI | eGFR | 53.7 | 26.7 | 4.8 | 31.0 | 14.0 | 30.7 | 4.9 | 9.4 |

| | | | | | | | | |
|---|---|---|---|---|---|---|---|---|
| AKI cause | Dehydration | Rapidly | Acute | Nephrotic | Dehydration, | Dehydration | Nephrotic | Rapidly |
| | | progressive | glomerular | syndrome, | blood | | syndrome, | progressive |
| | | glomerulo- | nephritis | blood | pressure | | blood | glomerulo- |
| | | nephritis | | pressure | drop | | pressure | nephritis |
| | | | | drop | | | drop | |
| Urine protein | ± | 3+ | 3+ | 4+ | − | − | 4+ | 2+ |
| Urine occult blood | − | 3+ | 3+ | ± | 1+ | − | 2+ | 3+ |
| Diabetes | − | − | + | − | + | − | − | − |
| D-Ala concentration | 1.2 | 1.7 | 8.5 | 3.9 | 10.7 | 0.9 | 18.4 | 1.5 |
| D/L-Ala ratio | 0.004 | 0.006 | 0.029 | 0.005 | 0.014 | 0.003 | 0.051 | 0.003 |

### Measurement of Blood D-Amino Acids

### Sample Preparation

Sample prepare from human blood plasma was carried out as follows: First a 20-fold volume of methanol was added to and completely mixed with the blood plasma. After centrifugation, 10 μL of supernatant obtained from the methanol homogenate was transferred to a brown tube and dried under reduced pressure. To the residue there were added 20 μL of 200 mM sodium borate buffer (pH 8.0) and 5 μL of fluorescent labeling reagent (40 mM 4-fluoro-7-nitro-2,1,3-benzooxadiazole (NBD-F) in anhydrous MeCN), and the mixture was then heated at 60° C. for 2 minutes. The reaction was suspended by addition of 75 μL of aqueous 0.1% TFA (v/v), and 2 μL of the reaction mixture was supplied to two-dimensional HPLC.

### Quantitation of Amino Acid Optical Isomers by Two-Dimensional HPLC

The amino acid optical isomers were quantified using the following two-dimensional HPLC system. NBD derivatives of the amino acids were separated and eluted using a reversed-phase column (KSAA RP, 1.0 mm i.d.×400 mm; Shiseido Corp.), in the mobile phase (5 to 35% MeCN, 0 to 20% THF, 0.05% TFA). The column temperature was 45° C. and the mobile phase flow rate was 25 μL/min. The separated amino acid fraction was separated off using a multi loop valve, and optically resolved in a continuous manner with a chiral column (KSAACSP-001S, 1.5 mm i.d.×250 mm; Shiseido Corp.). The mobile phase used was a MeOH/MeCN mixed solution containing citric acid (0 to 10 mM) or formic acid (0 to 4%), according to the amino acid retention. NBD-amino acids were detected by fluorescence detection at 530 nm using excitation light of 470 nm. The NBD-amino acid retention time was identified from standard amino acid optical isomers and quantified by a calibration curve.

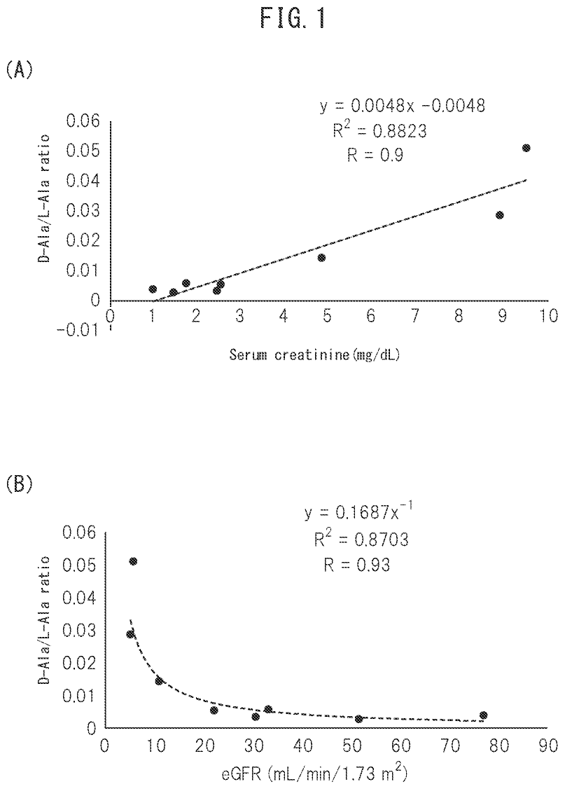

The serum creatinine and D-alanine/L-alanine ratio during AKI were plotted on a scatter plot, and the correlation coefficient was found to be R=0.9 (FIG. 1A). Also, the estimated glomerular filtration rate determined from the serum creatinine during AKI, and the D-alanine/L-alanine ratio, were plotted on a scatter plot, and the correlation coefficient was found to be R=0.93 (FIG. 1B). In addition, the serum creatinine and D-alanine levels were plotted on a scatter plot, and the correlation coefficient was found to be R=0.8 (FIG. 2A). The estimated glomerular filtration rate determined from serum creatinine during AKI, and the D-alanine level, were also plotted on a scatter plot, and the correlation coefficient was found to be R=0.93 (FIG. 2B). Based on these results it is concluded that blood D-alanine/L-alanine ratio and D-alanine level can be used as markers similar to serum creatinine, which is a marker for kidney damage in critical phase.

1. A marker for assessment of critical phase kidney damage, which is a marker value based on blood D-alanine level or D-alanine level and L-alanine level.

2. The marker according to claim 1, wherein the marker value based on D-alanine level and L-alanine level is a ratio or percentage.

3. The marker according to claim 1, wherein critical phase kidney damage is assessed for a patient under surgery or intensive care.

4. The marker according to claim 1, wherein the patient under surgery or intensive care is in a state selected from the group consisting of dehydration, nephrotic syndrome, glomerular nephritis, rapidly progressive glomerulonephritis and blood pressure drop.

5. A blood analysis method for a patient under surgery or intensive care, wherein the blood analysis method comprises:
a step of measuring blood D-alanine level or D-alanine level and L-alanine level, and
a step of associating a marker value based on the D-alanine level or the D-alanine level and L-alanine level with critical phase kidney damage.

6. The blood analysis method according to claim 5, wherein the marker value based on D-alanine level and L-alanine level is a ratio or percentage.

7. The blood analysis method according to claim 5, wherein
the patient under surgery or intensive care is in a state selected from the group consisting of dehydration, nephrotic syndrome, glomerular nephritis, rapidly progressive glomerulonephritis and blood pressure drop.

8. The blood analysis method according to claim 5, which is for specifying the stage or pathological condition in the critical phase by combining renal function markers.

9. The blood analysis method according to claim 8, wherein the renal function marker is at least one marker selected from the group consisting of urine NGAL, blood NGAL, urine IL-18, urine KIM-1, urine L-FABP, blood creatinine, urine creatinine, blood cystatin C, urine protein, urine albumin, urine β2-MG, urine α1-MG, urine NAG, eGFR (creatinine, cystatin C) and blood urea nitrogen.

10. A blood analysis system for assessing critical phase kidney damage for a patient under surgery or intensive care, comprising a storage unit, an analytical measurement unit, a data processing unit and a pathological information output unit, wherein:
the storage unit stores a threshold for assessment of critical phase kidney damage,
the analytical measurement unit separates and quantifies the blood D-alanine level or the D-alanine level and L-alanine level,
the data processing unit compares the marker value for the D-alanine level or the D-alanine level and L-alanine level of the patient with the threshold stored in the storage unit, and assesses critical phase kidney damage, and
the pathological information output unit outputs information relating to critical phase kidney damage.

11. The blood analysis system according to claim 10, wherein the marker value based on D-alanine level and L-alanine level is a ratio or percentage.

12. The blood analysis system according to claim 10, wherein the patient under surgery or intensive care is in a state selected from the group consisting of dehydration, nephrotic syndrome, glomerular nephritis, rapidly progressive glomerulonephritis and blood pressure drop.

13. A program which causes an information processing device comprising an input unit, an output unit, a data processing unit and a storage unit to determine critical phase kidney damage, wherein the program includes commands to cause the information processing device to:
cause the storage unit to store a calculation formula for a marker value inputted through the input unit, and a threshold for the marker value,
cause the storage unit to store a blood level of D-alanine or of D-alanine and L-alanine that has been inputted through the input unit,
cause the data processing unit to read the stored blood levels of D- and L-alanine and the calculation formula for the marker value and calculate the marker value, and cause the storage unit to store them, and
cause the data processing unit to read the stored marker value and the marker value threshold and compare the marker value with the threshold, and to output the presence or absence of critical phase kidney damage to the output unit.
